(19) United States
(12) Patent Application Publication
(10) Pub. No.: US 2011/0137223 A1
(43) Pub. Date: Jun. 9, 2011

(54) Sports bandage and improved flexible medical devices

(76) Inventor: M. Tyson Daniel, Roanoke, VA (US)

(21) Appl. No.: 12/960,193

(22) Filed: Dec. 3, 2010

Related U.S. Application Data
(60) Provisional application No. 61/266,357, filed on Dec. 3, 2009.

(51) Int. Cl. A61L 15/00

(52) U.S. Cl. 602/76

(57) ABSTRACT

The present invention relates to the field of flexible medical devices. More particularly, the present invention relates to flexible fabric bandages comprising inter-woven stretchable material with anti-slip characteristics and storage devices for protecting the bandages while not in use, for administering the bandages during use, and for re-packaging the bandages after use. In an embodiment, the present invention provides a flexible medical device comprising: a base fabric woven of elastic material and of a weave operably configured to provide a fabric having an upper surface and a lower surface and which is capable of being stretched longitudinally and laterally; and elastic silicone thread interwoven into the base fabric in a manner that provides the silicone thread exposed longitudinally along the lower surface of the fabric.

### CROSS-REFERENCE TO RELATED APPLICATIONS

This application relies on the disclosure of and claims priority to and the benefit of the filing date of U.S. Provisional Application No. 61/266,357, filed on Dec. 3, 2009, which is hereby incorporated by reference herein in its entirety.

### BACKGROUND OF THE INVENTION

1. Field of the Invention

The present invention relates to the field of flexible medical devices. More particularly, the present invention relates to flexible fabric bandages comprising inter-woven stretchable material with anti-slip characteristics and storage devices for protecting the bandages while not in use, for administering the bandages during use, and for re-packaging the bandages after use.

2. Description of Related Art

Flexible medical devices, such as orthopedic compression bandages, braces and sleeves have long been employed to provide support for athletic and other medical purposes. They are commonly worn over the trunk, shoulders, wrists, elbows, knees and ankles. They are also frequently employed on the lower legs and forearms, and, on the upper legs and arms, shoulders and chest. They provide support during normal movement, which support may be especially required by persons recovering from previous injuries or by persons who are frail or elderly. Such flexible medical devices also provide support for ligaments, tendons, muscles and joints against the stresses of over extension which may occur during exercise. In this manner, flexible medical bandages help to prevent orthopedic and muscular injury or re-injury. Elastomeric sleeves have also been employed, commonly in the form of stockings, to provide compression in order to facilitate peripheral venous return from the legs of bed-bound patients, thereby helping to prevent embolism.

These flexible medical devices should have sufficient elasticity, or stretch, to enable them to conform to the bandaged area and when secured, to allow limited movement and swelling to take place so that circulation is not adversely affected.

Typical flexible medical devices include elastic adhesive bandages, which traditionally comprise natural yarns, woven to form the fabric. Normally the yarns are woven using a plain weave design. The woven fabrics of the prior art offer extension in the warp, or length, direction only but lack multi-directional extension. Traditionally natural fiber materials were used as these materials were seen as superior to synthetic materials. Although synthetic and/or elastomeric yarns may offer greater initial stretch and superior fatigue resistance than natural yarns, such as cotton, natural yarns have been traditionally preferred for flexible medical devices. This is due to the aesthetic appearance of natural yarns and the perceived greater control stretch value, or controlled compression, of natural yarns than synthetic and/or elastomeric yarns.

However, regardless of what types of yarns are used to produce a stretch fabric, the loss of fatigue resistance over time still remains an issue. In addition, commonly used flexible bandages and other medical devices have a skin-contacting layer that is typically quite slick, and the support tends to migrate along the skin unless it is sized and custom fitted to the limb of a wearer. This is especially true of supports placed around joints, such as the knee, as these supports slip or migrate along the leg.

Known knitted, woven and laminate materials tend to experience slippage along the limb and to wrinkle or bunch up, causing compression and/or coverage of the target area or limb to be uneven. Such shifting and uneven displacement of the material against the skin can cause dermal irritation and discomfort to the wearer. Slippage of supports can be limited, but not eliminated by custom fitting. However, such fitting requires personal consultation with a professional fitter. Thus, it is expensive and consequently unavailable to most users. Changes in body weight, weight distribution or development of musculature due to growth, exercise or aging may necessitate periodic refitting of the bandage or device to maintain proper support.

Because of these problems, some braces, bandages, and devices have been constructed of rubber-like polymeric materials such as neoprene, which tends to stay in place because of its high coefficient of friction against the skin. Neoprene supports are generally thicker and bulkier than counterparts constructed of other materials, and such materials have not proven to be satisfactory for frequent or sustained use because of their lack of permeability to air, water, and/or body fluids, such as perspiration. Supports constructed of neoprene do not permit the underlying skin of the wearer to breathe. Because such impermeable supports lack ventilation to carry away body heat and moisture, extended or frequent wear may be uncomfortable as well as irritating to the skin. If such irritation is prolonged, it can result in morbidity such as dermatitis and sloughing of the skin. Such impermeable materials are especially unsuitable for support devices to be worn by amputees or individuals with impaired circulation, who may develop necroses. In addition, since impermeable supports provide no outlet for perspiration excreted by the wearer, a salt residue may be deposited on the inner surface of the support which eventually impairs elasticity and shortens its effective lifespan.

None of the previously available materials and combinations of materials provides effective elastomeric support and compression while staying in place and maintaining breathability for the underlying skin surface. Accordingly, there is a need for a flexible medical device, including a sports bandage, for athletic and medical uses which is light weight, comfortable, and stretchable to conform to the anatomy of a user and to permit movement with increased flexibility, which resists shifting against the skin and migration during exercise and which does not impair breathability of the underlying skin or circulation of the underlying blood vessels, and which has greater fatigue resistance.

### SUMMARY OF THE INVENTION

The numerous limitations inherent in the flexible medical devices described above provide great incentive for new, better sport bandage systems, devices, and methods capable of accounting for one or more of these issues. The present invention relates to flexible fabric bandages and devices for protecting, administering and re-packaging such bandages.

Embodiments of the invention include flexible medical devices comprising: a base fabric woven of elastic material and of a weave operably configured to provide a fabric having an upper surface and a lower surface and which is capable of being stretched longitudinally and laterally; and elastic silicone thread interwoven into the base fabric in a manner that provides the silicone thread exposed longitudinally along the lower surface of the fabric; and wherein during use the flexible medical device is capable of longitudinal stretching of from about 90% to about 120% of its length and is capable of lateral stretching of from about 5% to about 20% of its width.

Other embodiments provide a device for administering and storing a flexible medical bandage device comprising: a housing operably configured to protect the flexible bandage when not in use; a spool for wrapping the bandage; and a spooling mechanism for allowing the bandage to be rolled onto the spool within the housing; wherein the flexible bandage comprises a base fabric woven of elastic material and of a weave operably configured to provide a fabric having an upper surface and a lower surface and which is capable of being stretched longitudinally and laterally; and elastic silicone thread interwoven into the base fabric in a manner that provides the silicone thread exposed longitudinally along the lower surface of the fabric. Such devices allow for easy storage, administration and re-storage of the flexible bandages described herein.

The features of novelty and various other advantages that characterize the invention are pointed out with particularity in the claims forming a part hereof. However, for a better understanding of the invention, its advantages, and the objects obtained by its use, reference should be made to the drawings that form a further part hereof, and to the accompanying descriptive matter, in that there is illustrated and described a preferred embodiment of the invention. The features and advantages of the present invention will be apparent to those skilled in the art. While numerous changes may be made by those skilled in the art, such changes are within the spirit of the invention.

### BRIEF DESCRIPTION OF THE DRAWINGS

These drawings illustrate certain aspects of some of the embodiments of the present invention, and should not be used to limit or define the invention.

FIG. 1 is a top perspective view of a schematic drawing of an exemplary flexible bandage comprising a silicon weave according to embodiments of the invention.

FIG. 2 is a top left perspective view of a schematic drawing of a roll of an exemplary bandage according to the invention, e.g., a Sports Bandage wrap, comprising a silicon weave longitudinally along the left and right sides of the bandage.

FIG. 3 is side perspective view of a schematic diagram of a sports bandage embodiment of the present invention comprising a silicone weave.

FIG. 4 is a front perspective view of a schematic diagram of the wrap of FIG. 2, illustrating the silicon weave on the left and right sides of one surface of the bandage.

FIG. 5 is a close up top left perspective view of a schematic drawing of the wrap of FIGS. 2 and 4, showing several rows of the silicon material weaved longitudinally into the top surface of the fabric bandage.

FIG. 6 is a close up side perspective view of a schematic drawing of the wrap of FIGS. 2 and 4, illustrating an exemplary pattern for the longitudinal silicon weave.

FIG. 7 is a perspective view of a schematic drawing of a bandage embodiment according to the present invention.

FIG. 8 is a photograph of a bandage embodiment of the invention showing a close up top planar view of the material comprising the silicon weave (above) incorporated and woven into a single face of the wrap.

### DETAILED DESCRIPTION OF PREFERRED EMBODIMENTS

In accordance with embodiments of the present invention, the present invention is directed to improved flexible medical devices for use in treatment and prevention of injuries in patients. More particularly, the present invention relates to flexible fabric bandages and devices for protecting, administering and re-packaging such bandages.

One of the many potential advantages of the methods of the present invention, only some of which are discussed herein, is that the invention provides improved bandaging systems that allow for more efficient protection of the areas intended for treatment. Bandages according to the present invention tend not to slip once placed on the skin or resist slippage with respect to the surface to which the bandage is applied, e.g., skin. The bandages described herein are capable of conforming to a surface area even over joints or other asymmetrical areas. In some embodiments, the bandages provide better and more secure compression leading to improved treatment or prevention of injury. The specialized elasticity and compressibility features of bandages described in the embodiments according to the invention provide many advantages including, but not limited to, the ability of the bandages to preferentially not adhere to hair, skin, and clothing.

Certain embodiments of the invention include flexible medical devices comprising: a base fabric woven of elastic material using a weave pattern operably configured to provide a fabric having an upper surface and a lower surface and which is capable of being stretched longitudinally and laterally. Certain embodiments further provide an elastic silicone thread interwoven into the base fabric of such bandages in a manner that provides the silicone thread exposed longitudinally along the lower surface of the fabric. Preferably, the silicone thread is exposed only on one side of the fabric. In certain embodiments, the flexible medical devices described herein may be capable of longitudinal stretching in the range of from about 90% to about 120% of their length and may be capable of lateral stretching of from about 5% to about 20% of their width. In the context of this application, the degree of stretching refers to the additional amount the material will lengthen due to stretching.

Certain embodiments of the invention include fabric flexible medical devices that may be any length and width and may be stretchable in at least one direction. The length and width of the flexible medical devices may vary depending on their particular use. One of ordinary skill in the art, with the benefit of this disclosure, would know the right length and width needed for a particular application. Preferred embodiments include flexible medical devices with fibers woven to provide a flexible medical device that is stretchable in both the warp (longitudinal) and weft (lateral) directions, or any direction in between. In certain embodiments, the flexible medical devices may be stretchable in at least two directions. In other embodiments, the flexible medical devices may be stretched in a plurality of directions.

The flexible medical devices described in the present invention include, but are not limited to, bandages, waistbands, sock bands, wrist bands, head bands, stockings, pantyhose, and footwraps to name a few, such as those disclosed in U.S. Pat. No. 5,865,779, the disclosure of which is hereby incorporated by reference herein in its entirety, and any other related medical devices capable of being adapted to include features of the invention.

Any weave pattern capable of providing a stretchable fabric can be used in the flexible medical device embodiments of the invention, including weaves that combine the weft and/or warp threads at anywhere from almost parallel to 90 degrees with respect to one another, and especially in a criss-cross pattern or other weaving pattern that positions some of the threads at about 45 degrees relative to other threads in the weave.

Numerous weave patterns for fabric are known. Any weave pattern that provides a stretchable fabric can be used in the bandages according to the invention. A person of ordinary skill in the art would know the appropriate weave pattern to choose depending on the desired application. Certain known weaves that can be used with the flexible medical devices described herein include, but are not limited to, plain weave, twill weave, basket weave, leno weave, satin weave, and any combination thereof. These types of weaves are described in Table 1 below.

TABLE 1

| Type of | |
|---|---|
| Weave | Description |
| Plain | A plain weave is one of the most simple weave patterns. |
| | The warp and filling threads cross alternately. Plain-woven |
| | fabrics are generally the least pliable, but they are the most |
| | stable. Characteristics of a fabric having a plain weave |
| | include high particle retention and low resistance to |
| | blinding, with average cake release properties. |
| Twill | The twill type weave pattern is characterized by a diagonal |
| | rib, or twill line. Each warp floats over at least two or more |
| | consecutive fill yarns, enabling a greater number of yarns |
| | per unit area than a plain weave, while not losing a great |
| | deal of fabric stability. Characteristics of the twill include |
| | average resistance to blinding, average cake release, and |
| | good mechanical strength. |
| Basket | A variation of the plain weave where two or more warp |
| | yarns cross alternately with two or more filling yarns is the |
| | basket weave, which resembles a plaited basket. This |
| | weave is typically more pliable and stronger than a plain |
| | weave, but is looser and not as stable. The basket weave is |
| | typically used for backing cloth. |
| Leno | The leno weave is a locking type weave in which two or |
| | more warp threads cross over each other and interlace |
| | with one or more filling threads. It is used primarily to |
| | prevent shifting of fibers in open weave fabrics and usually |
| | only for backing (support) cloths. |
| Satin | The face of a fabric having a satin weave consists almost |
| | completely of warp produced in the repeat of the weave. |
| | This is the most flexible of weave patterns and conforms |
| | very easily around most contoured surfaces. Satin weaves |
| | are usually four, five, eight, or twelve harness. |
| | Characteristics of a satin type weave include for example |
| | excellent cake release, average retention, and excellent |
| | resistance to blinding. |

In certain embodiments, one or more patterns of weaves can be interwoven into the fabric to provide a more complex weave with greater stretching characteristics. The flexible medical devices can be single- or multi-ply. Various elastic flexible medical devices and/or elastic woven fabrics are known and can be used in certain embodiments of the invention, including those disclosed for example in U.S. Pat. Nos. 889,827; 2,823,444; 3,622,431; 4,424,808; 3,409,008; 3,618,754; and 4,207,885, each of which is hereby incorporated by reference herein in its entirety.

An embodiment of the flexible medical devices according to the invention includes, but is not limited to, any known flexible medical device, such as a bandage, modified in such a way to replace or add one or more longitudinal threads of the fabric with a non-slip material such as silicone. In preferred embodiments, the silicone thread is woven or inter-woven into one side of the flexible medical device. For example, the silicone can be woven into the base fabric in a manner to provide the top surface (or the side of the bandage that faces away from skin) as one material or a composite material and the bottom surface (side of flexible medical device that contacts skin) as a material or composite material plus the silicone protruding from the material.

Some embodiments comprise flexible medical devices that are highly longitudinally and laterally elastic, or highly longitudinally and slightly less laterally elastic, or highly laterally and slightly less longitudinally elastic. Such embodiments can be designed to fit particular needs, such as flexible medical devices with high compression capability within a concentrated area whether more or less in a lateral or longitudinal direction.

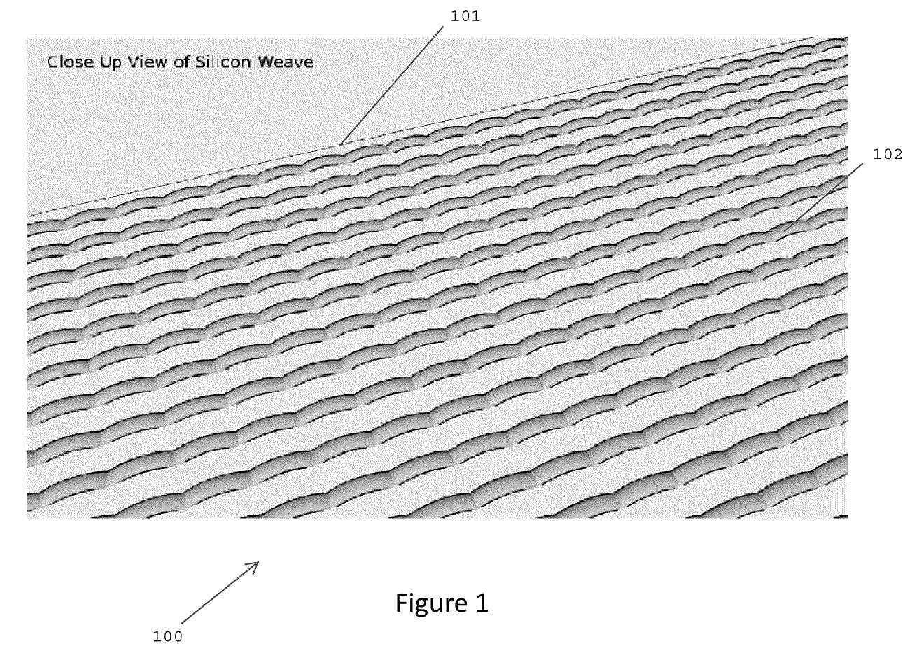

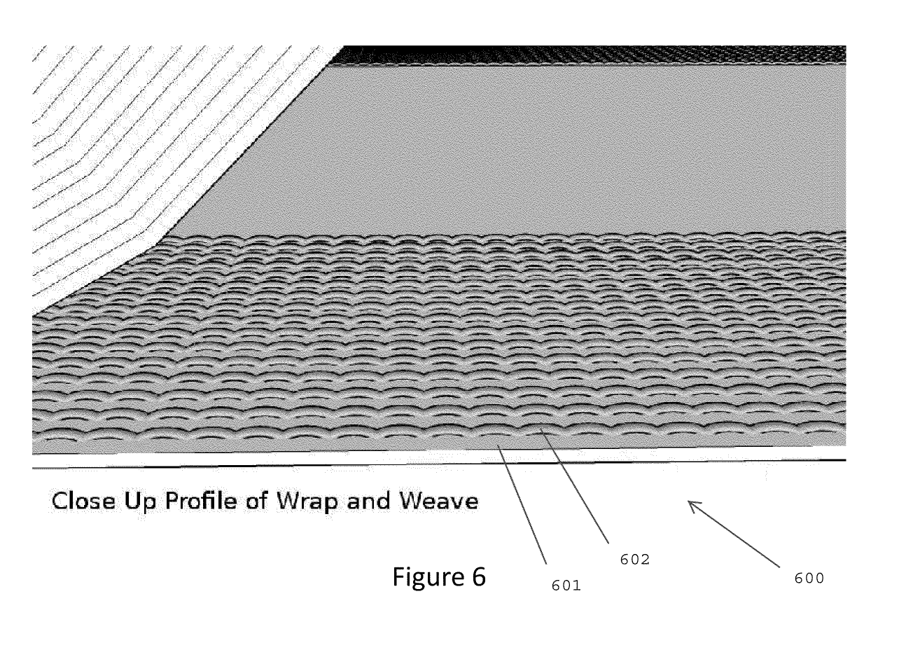

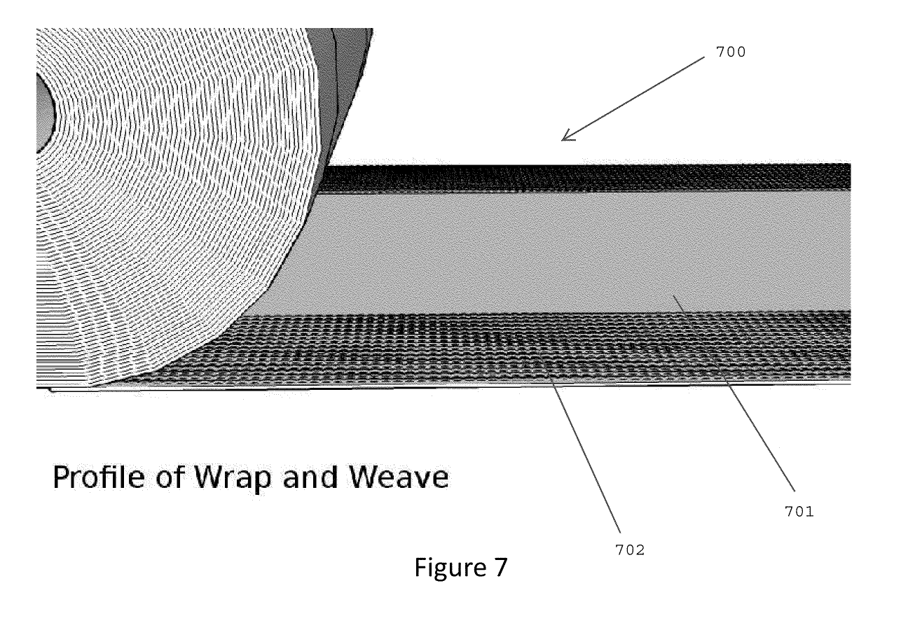

In an embodiment, the flexible medical devices may be bandages. FIGS. 1-8 provide examples of representative bandages according to the invention. As shown in these figures, embodiments of the invention can generally comprise a sports bandage comprising: a base fabric having a first and second surface, which is woven of elastic material and is capable of expanding longitudinally in length and laterally in width; and elastic silicone thread interwoven into the base fabric in a manner that provides the silicone thread exposed longitudinally along a portion of the first surface of the fabric; and wherein, when stretched, the fabric is capable of longitudinal expansion ranging from about 90% to about 120% of its resting length and is capable of lateral expansion ranging from about 5% to about 20% of its resting width. FIG. 1 provides a close-up view of an exemplary bandage 100 according to the invention that comprises a base material 101 with silicone threads 102 incorporated therein.

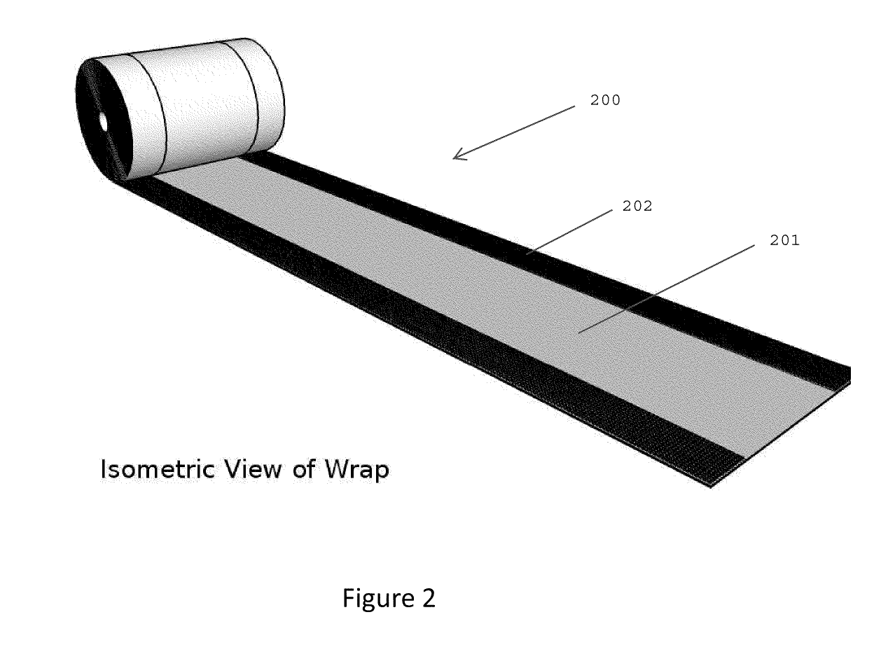

A preferred embodiment is a sports bandage that may be in the range of from about 4 to about 6 inches in width and in the range of from about 10 to about 50 feet in length, which bandage 200 is illustrated in a partially rolled configuration in FIG. 2. In some embodiments, the length of the bandages may be in the range of from about 10 to about 20 feet in length. Such bandages can be used to wrap large sections of the body for example several times around a person's trunk or the combination of a person's trunk and shoulder. As further shown in FIG. 2, the silicone threads 202 can be incorporated into the base fabric 201 of bandage 200 by being stitched into, on or through the base fabric weave 101 in a direction that runs lengthwise along the edges of the bandage 200, leaving the center portion or middle section of the bandage with only the base fabric material. In alternative embodiments, the silicone threads can be incorporated into the base fabric width-wise and/or along an entire surface of the bandage instead of just along the edges.

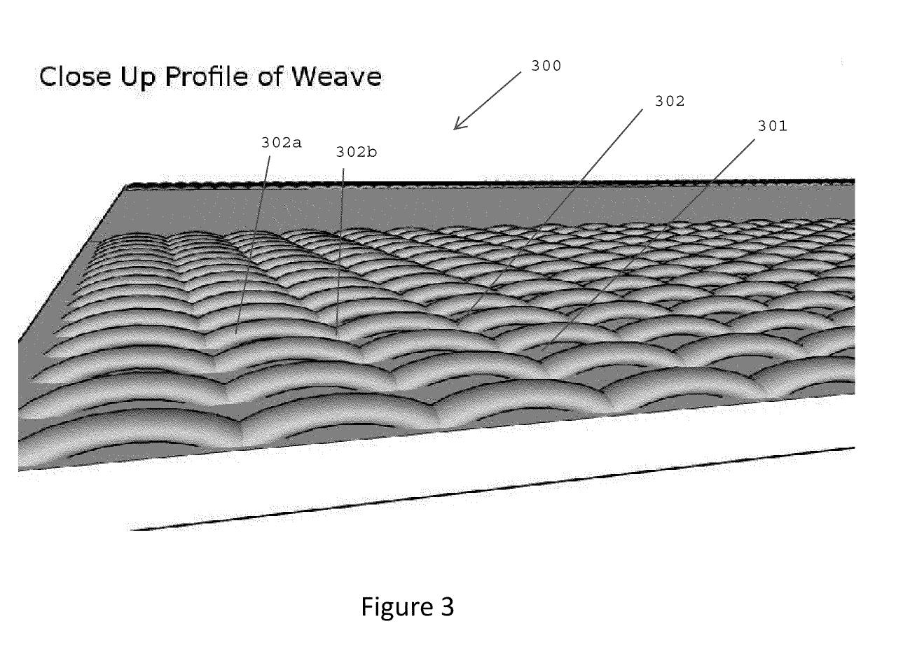

In FIG. 3, an embodiment of a flexible medical device 300 comprising a base material 301 and silicon thread stitching 302 is provided. As shown, the silicone thread can be arranged in several substantially parallel rows along the bandage 300 longitudinally (as shown) and/or laterally (along the width, not shown). The stitching can be lined up (as shown) in a manner that provides the loops 302a of the silicone thread 302 in line with other silicone thread loops of parallel rows of the thread, as well as providing the stitched portion 302b in line with the stitched portion of the other rows. It is not critical how the silicone thread 302 of one row is arranged relative to another row or relative to the stitching or weave of the base fabric.

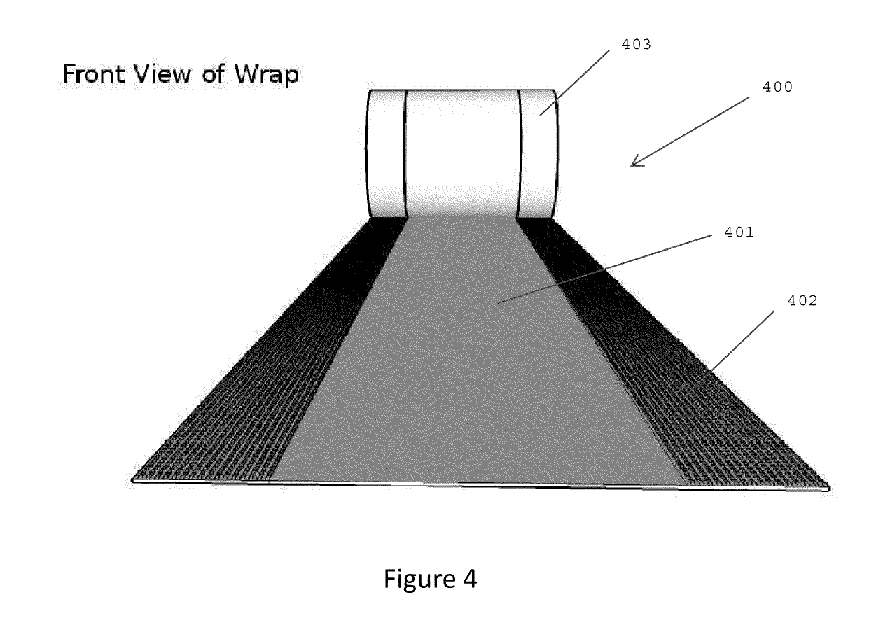

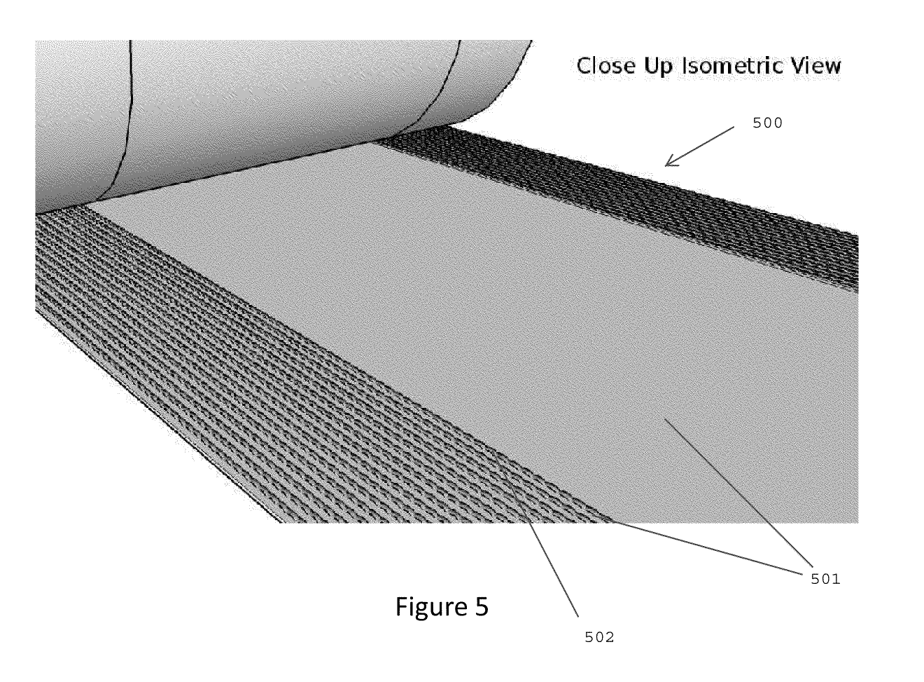

FIG. 4 provides another view of a bandage 400 according to the invention, which is shown in a rolled 403 configuration and comprises the base material 401 in the middle portion of the inside surface of the bandage 400 as well as on the entire outside surface of bandage 400, with the silicone thread 402 stitching longitudinally stitched along the length of the bandage 400. FIGS. 5, 6, and 7 shows bandage 500, 600, and 700 at a slightly different angles to emphasize the detail of the stitching of silicone threads 502, 602, and 702 into, on and/or through the base material 501, 601, and 701.

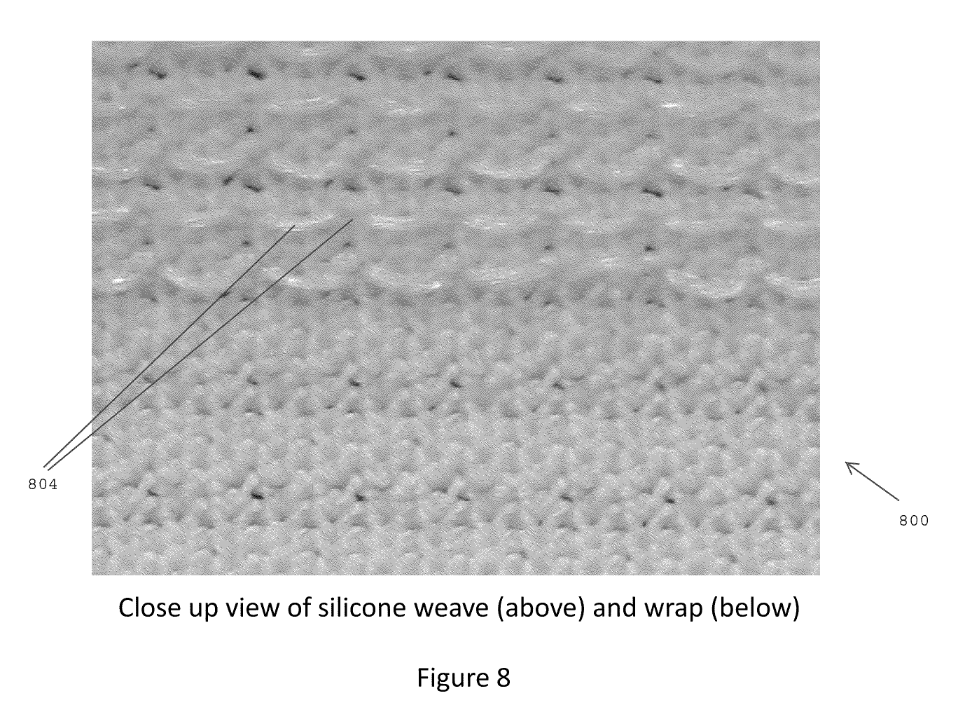

Even further, FIG. 8 provides a close-up view of an inside surface of bandage 800 to illustrate stitching 804, which comprises the silicone threads in a running stitch pattern which helps contribute to capability of bandage 800, when subject to stretching, to be capable of longitudinal expansion ranging from about 90% to about 120% of its resting length and capable of lateral expansion ranging from about 5% to about 20% of its resting width. Having the silicone thread incorporated into the base fabric in this manner will not limit the stretchability of the base material. As shown, the filicone threads can be loosely incorporated into the base material to further facilitate and/or not interfere with the expansion capabilities of the base material or the overall bandage 800.

The invention includes flexible medical devices that comprise a stretchable base fabric comprising a fabric woven of elastic fibers or woven in such a way as to impart elasticity or more elasticity to the bandage other than from the characteristics of the fibers alone. The base fibers and/or weave can have or contribute an elongation of up to about 300% in the fibers and/or flexible medical device. Especially preferred are flexible medical devices capable of stretching from about 50% to about 300% in length and/or width when compared to the original resting state of the flexible medical device. Fabrics that can be used as the base fabric include, but are not limited to, cotton, flax, wool, linen, silk, nylon, polyester, spandex, lycra, viscose, rayon staple, and any combinations, composites, and blends of these materials. The nature of the fabric is not critical, so long as a lightweight, flexible, soft, and comfortable flexible medical device results. If used for other fabric applications, one of ordinary skill in the art would be familiar with various modifications that can be made to suit such applications.

In certain embodiments of the present invention, a gripping material may be incorporated, woven, included, comprised in or on, adhered to, or combined as a composite of the base material, or the fabric of the base material, to the flexible medical devices to provide means for gripping a surface to which the device contacts, such as skin. With respect to bandages in particular, it is preferred that the gripping material not be an adhesive and be capable of being removed from a surface with little to no effort. Other uses, however, may benefit from the gripping material having adhesive characteristics. It is further preferred that the base bandage material and the gripping material have elasticity in that they are capable of returning to about the length or width of their original state after being stretched. Of course, over time and as a result of repeated stretching or continuous stretching for substantial periods of time, elasticity of certain fabrics may be reduced. Thus, for bandage-type applications it is desired that the material have some degree of rebound from a stretched state.

The gripping material can be threads of non-slip material inter-woven into the base fabric. The stitching technique used to introduce the non-slip material into the base fabric can be performed in numerous ways, including by intra-looping (e.g., passing of a loop of thread through another loop formed by the same thread), interloping (e.g., passing of a loop of thread through another loop formed by a different thread, or interlacing (e.g., passing of a thread over or around another thread or loop of another thread) to name a few. For example, silicone thread or yarn can be inter-woven into the base fabric by looping the silicone over and partially through one or more layers, threads, or weave of the fabric. The silicone can pass through the base fabric and be exposed on either side or both sides of the bandage, which would render either side of the bandage capable of being used, e.g., a reversible bandage. Alternatively, the silicone or other non-slip material can be woven into and through the base fabric in such a manner that the silicone is exposed only on one side of the bandage. A preferred stitch for incorporating the silicone thread into the base fabric is a running stitch which may or may not pass completely through both sides of the fabric or through one layer of a double woven fabric. It is especially preferred to have several substantially parallel rows of silicone thread stitched or woven into the fabric and extending along the length of the bandage.

A preferred embodiment includes the non-slip material longitudinally along the length and near the longitudinal edges of the base fabric, leaving the middle section of the interior side of the bandage as comprising only the base fabric for the material. Preferred are flexible medical devices comprising materials and/or woven in such a way as to provide elongation of about 150% along the edges and of about 110% in the middle longitudinal section of the bandage.

The non-slip material is preferably in the form of fibers, such as a silicone thread. The fibers can be arranged on, in, and/or inter-woven with the base fabric. Several select strands of the base fabric can also be coated with the non-slip material(s). Many non-slip materials exist which can be included in embodiments of the invention, including, but not limited to, rubber latex, silicone, and elastic.

Other embodiments include non-slip materials that are more elastic than the base materials to provide a more stretchy fabric than without the material. Greater stretchability can be achieved by substituting one or more of the longitudinal or lateral fibers of the flexible medical device with a material having higher elasticity and non-skid capabilities. When such materials are inter-woven into or within the vicinity of the edges, the edges may be capable of stretching more than the main part (or inner middle section) of the flexible medical device. Additionally, some embodiments can comprise the non-slip material or highly elastic material arranged laterally with respect to the bandage.

The flexible medical devices may include one or more means for securing the bandage in place on the body, including metal clasps or grips operably configured to grip a surface of the end of the bandage and a surface of a section of the wrapped bandage to hold the two portions of the bandages together at their respective surfaces. Other types of grips can include Velcro (hook and loop type fasteners) strategically placed on the ends of the flexible medical device to keep them in place during use. Such means for securing the bandage to itself can include Velcro or other fasteners located at desired predetermined positions on a surface along the length of the bandage.

In the bandage embodiments of the invention, a device for administering the bandage comprising: a housing operably configured to protect the bandage when not in use; a spool for wrapping the bandage; a spooling mechanism for allowing the bandage to be rolled onto the spool within the housing is disclosed herein. In preferred embodiments, the bandages can be washed and re-used, reducing bandage waste. The bandages may also be disposable.

Preferably, the non-skid fibers are positioned with respect to the base fabric of the flexible medical devices in such a way so that the fibers contact the skin at a plurality of contact points. One embodiment provides the silicone fibers as rounded fibers which are oriented in the base fabric so that skin will contact up to 50% or more of the circumference of the exposed portion of the non-skid fiber. When stretched over the skin surface the bandage has a tendency to pull on the skin and compact the surface of the skin between the fibers.

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Other embodiments of the invention will be apparent to those skilled in the art from consideration of the specification and practice of the invention. It is intended that the specification and examples be considered as exemplary in nature and that variations that do not depart from the essence of the invention are intended to be within the scope of the invention.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While devices, systems, and methods are described in terms of “comprising,” “containing,” or “including” various components or steps, the devices, systems, and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

1. A flexible sports bandage comprising:
a base fabric having a first and second surface, which is woven of elastic material and is capable of expanding longitudinally in length and laterally in width; and
elastic silicone thread interwoven into the base fabric in a manner that provides the silicone thread exposed longitudinally along a portion of the first surface of the fabric; and
wherein, when stretched, the fabric is capable of longitudinal expansion ranging from about 90% to about 120% of its resting length and is capable of lateral expansion ranging from about 5% to about 20% of its resting width.

2. The sports bandage of claim 1, wherein the base fabric is selected from cotton, flax, wool, linen, silk, nylon, polyester, spandex, lycra, viscose, rayon staple, and combinations, composites, and blends thereof.

3. The sports bandage of claim 1, wherein the base fabric comprises a plain, twill, basket, leno, or satin weave, and any combination thereof.

4. The sports bandage of claim 1, operably configured to comprise a waistband, a sock band or sock, a wrist band, a head band, a stocking, pantyhose, or a footwrap.

5. The sports bandage of claim 1, wherein the flexible medical device is capable of stretching in a plurality of directions in addition to lateral and longitudinal expansion.

6. The sports bandage of claim 1 which is washable and re-usable.

7. The sports bandage of claim 1, wherein the elastic silicone thread is disposed in several rows of stitching extending along the length of the bandage in a longitudinal pattern.

8. The sports bandage of claim 1, having a length in the range of from about 10 feet about to about 50 feet.

9. The sports bandage of claim 8, having a width in the range of from about 4 inches to about 6 inches.
